(19) United States
(12) Patent Application Publication
(10) Pub. No.: US 2024/0035654 A1
(43) Pub. Date: Feb. 1, 2024

(54) LIGHTING APPARATUS USING PCB BOARD IN WHICH VISIBLE-LIGHT STERILIZATION LED AND LED HAVING INFRARED WAVELENGTH ARE COMBINED

(71) Applicant: Daons Co. Ltd., Goyang-si Gyeonggi-do (KR)

(72) Inventor: Jong Jin KIM, Namyangju-si Gyeonggi-do (KR)

(73) Assignee: Daons Co. Ltd., Goyang-si Gyeonggi-do (KR)

(21) Appl. No.: 17/630,273

(22) PCT Filed: Dec. 3, 2021
(86) PCT No.: PCT/KR2021/018195

§ 371 Date: Jan. 26, 2022

(30) Foreign Application Priority Data
Nov. 26, 2021 (KR) 10-2021-0166142

(51) Int. Cl. F21V 33/00; F21V 23/04; A61L 2/10

(52) U.S. Cl. CPC F21V 33/0064; F21V 23/0471; A61L 2/10; F21Y 2115/10

(57) ABSTRACT

The present invention relates to a lighting apparatus using a PCB substrate in which a sterilization LED for emitting a light within a visible light region and an LED having an infrared wavelength are combined, in which a colored light is emitted while minimizing a wavelength of a blue light, so that the lighting apparatus is harmless to a human body, and has a sterilization effect. To this end, the present invention includes a PCB substrate on which a UV LED package including a first UV LED package including at least one first LED chip for emitting a light having a center wavelength of 405 nm and a fluorescent substance layer formed on the first LED chip, and configured to emit a colored light, and a second UV LED package, and an infrared LED package are disposed. When the lighting apparatus according to the present invention is used, while the colored light illuminates an inside of a room, a damage caused by a blue light is reduced, and a COVID-19 virus is effectively killed due to a sterilization effect. When lighting installed in public transportation and publicly used facilities is replaced with the lighting apparatus according to the present invention, the sterilization effect is naturally achieved while the lighting is provided, so that the spread of a COVID-19 virus is reduced.

### TECHNICAL FIELD

The present invention relates to a lighting apparatus using a PCB substrate in which a visible-light sterilization LED, which is harmless to a human body and configured to emit a light within a visible light band, such as a white, green, orange, or yellow light, and an infrared wavelength are combined. More specifically, the present invention relates to a lighting apparatus in which an LED element having a visible light wavelength and a sterilization effect is coated with a fluorescent material to emit a colored light while minimizing a blue light, which is a light having a blue color, so that the lighting apparatus is harmless to an eye and a human body, and has a sterilization effect.

### BACKGROUND ART

A light emitting diode (LED) is an inorganic light source, and has been rapidly utilized in various fields such as a display device, a vehicle lamp, and general lighting. In particular, since the LED has a long lifespan and low power consumption, the LED has rapidly replaced existing lighting sources.

Meanwhile, an ultraviolet (UV) light is well known to have sterilization and disinfection effects. Accordingly, various light sources having a sterilization function have been developed by using an ultraviolet LED. Related art document 1 (Korean Patent Registration No. 10-2300679) discloses a disinfector using an LED ultraviolet sterilization lamp, which is an invention utilizing such an ultraviolet LED. However, since the UV light is harmful to a human body, there is a restriction on the use of the UV light. This is because a disinfection mechanism of the UV light having a wavelength of 260 to 290 nm is a scheme that destroys DNA by cracking DNA molecules. For this reason, an LED sterilization scheme using the UV light may not be suitable to be used in lighting devices in a space where activities of people occur.

Meanwhile, in order to fabricate an LED that emits a white light, there is a scheme of implementing a white color by applying a fluorescent substance that emits a yellow light to a blue LED in which a blue light of the blue LED is used as an excitation source, and mixing the blue light emitted from the element with the yellow light emitted from the fluorescent substance. In other words, an LED element for emitting a white light generally adopts a scheme of applying a fluorescent substance to an LED to use a blue light emitted from the element and a secondary light source emitted from the fluorescent substance. Related art document 2 (Korean Patent Registration No. 10-1432479) discloses an invention of fabricating a light emitting diode that emits a white light by applying a yellow fluorescent substance to a blue light emitting diode. However, such a method of fabricating a light emitting diode of a white light is configured such that, in order to allow a blue LED to emit a white light, an element having a visible light wavelength band of 450 nm, which is the cheapest, is coated with a fluorescent material to emit the white light.

Recently, with the spread of COVID-19, facilities requiring sterilization and disinfection for prevention of epidemics have been increased, and the sterilization and disinfection have become increasingly essential for publicly used facilities or public transportation. In this situation, if a sterilization effect can be achieved with lighting used in the publicly used facilities or the public transportation, manpower and resources required for the prevention of epidemics may be saved, and prevention of the spread of COVID-19 may be assisted. However, since the publicly used facilities or the public transportation consistently have a floating population, it is difficult to use the above-described ultraviolet LED, and an LED for emitting a white light, which is generally used, rarely has a sterilization effect.

### Documents of Related Art

Related art document 1: Korean Patent Registration No. 10-2300679 (registered on Sep. 3, 2021)
Related art document 2: Korean Patent Registration No. 10-1432479 (registered on Aug. 14, 2014)

### DISCLOSURE

### Technical Problem

In order to solve the above problems, a technical object of the present invention is to provide a lighting apparatus using a PCB substrate, in which an LED having an appropriate center wavelength is used to obtain a sterilization function that is harmless to a human body.

In addition, a technical object of the present invention is to provide a lighting apparatus in which an LED for emitting a light that minimizes a blue light is provided to obtain a sterilization function without causing an eye disease, and a PCB substrate constituting the lighting apparatus.

In addition, a technical object of the present invention is to provide a lighting apparatus that allows various modes to be used as necessary through a switch for switching the lighting apparatus into the various modes.

In addition, a technical object of the present invention is to provide a lighting apparatus in which LEDs for emitting lights having various temperatures and colors within a visible light region, such as white, green, orange, and yellow lights, are provided to obtain a sterilization function that may be utilized in various environments.

### Technical Solution

The present invention provides a lighting apparatus including: at least one UV LED package including a first LED chip for emitting a light having a center wavelength of 400 to 410 nm and a fluorescent substance layer formed on the first LED chip, and configured to emit a light within a visible light band; and a PCB substrate on which the UV LED package is disposed.

In addition, the present invention provides the lighting apparatus further including at least one infrared LED package including a second LED chip for emitting a light having a center wavelength of 845 to 855 nm.

In addition, the present invention provides the lighting apparatus, wherein the light within the visible light band includes a light having one of a white color, a green color, an orange color, and a yellow color.

In addition, the present invention provides the lighting apparatus, wherein the UV LED package includes a first UV LED package and a second UV LED package, which have mutually different fluorescent substance layers to emit lights having mutually different colors.

In addition, the present invention provides the lighting apparatus further comprising a switch serving to cut off or connect a power supplied to the UV LED package and the infrared LED package.

In addition, the present invention provides the lighting apparatus further comprising a motion sensor, wherein the switch further includes an Auto-switch for operating the motion sensor to perform an operation according to motion detection of the motion sensor.

In addition, the present invention provides the lighting apparatus, wherein, in a case of using the Auto-switch, the power is supplied to automatically turn on lighting when the motion sensor detects a motion, and the power is automatically cut off to automatically turn off the lighting when the motion sensor does not detect the motion for a predetermined time.

In addition, the present invention provides the lighting apparatus, wherein, in a case of using the Auto-switch, when the motion sensor detects a motion, the first UV LED package is turned off after being turned on, and the second UV LED package is turned on after being turned off; and when the motion sensor does not detect the motion for a predetermined time, the first UV LED package is turned on again, the second UV LED package is turned off again, and the infrared LED package is continuously turned on regardless of whether the motion is detected.

In addition, the present invention provides the lighting apparatus, wherein, in a case of using the Auto-switch, the infrared LED package is turned on, and turned off when the motion sensor detects a motion, the infrared LED package is turned on again when the motion sensor does not detect the motion for a predetermined time, and the UV LED package is continuously turned on regardless of whether the motion is detected.

In addition, the present invention provides the lighting apparatus, wherein the PCB substrate is configured such that the UV LED package and the infrared LED package are arranged in three columns; the UV LED package is arranged in a first column and a third column, and the infrared LED package is arranged in a second column; and the UV LED packages arranged in the first and third columns are arranged in a same row, and the infrared LED package of the second column is arranged between rows of positions where the UV LED packages of the first and third columns are arranged so as to be alternately arranged with the UV LED packages of the first and third columns.

### Advantageous Effects

When the lighting apparatus according to the present invention is used, while lighting having various colors within the visible light band, such as white, green, orange, and yellow, and color temperatures illuminates an inside of a room, a damage caused by a blue light can be reduced, a sterilization effect can be obtained, and a COVID-19 virus can be effectively killed. Since there is always lighting in public transportation and publicly used facilities, which are usually crowded by the public, when the lighting installed in the public transportation and the publicly used facilities is replaced with the lighting apparatus according to the present invention, the sterilization effect may be naturally achieved while the lighting is provided, so that the spread of various pathogens and the COVID-19 virus can be reduced.

### DESCRIPTION OF DRAWINGS

FIG. 1 is a perspective view showing a lighting apparatus according to one embodiment of the present invention.

FIG. 2 is a view showing a UV LED package according to one embodiment of the present invention.

FIG. 3 is a view showing the UV LED package and an infrared LED package disposed on a PCB according to one embodiment of the present invention.

FIG. 4 is a right side view of the lighting apparatus showing a switch according to one embodiment of the present invention.

FIG. 5 is a photograph showing test results of a first antibacterial test of the UV LED package according to the present invention.

FIG. 6 is a table showing results of the first antibacterial test of the UV LED package according to the present invention.

FIG. 7 is a table showing results of a second antibacterial test of the UV LED package according to the present invention.

### BEST MODE

A lighting apparatus including: at least one UV LED package including a first LED chip for emitting a light having a center wavelength of 400 to 410 nm and a fluorescent substance layer formed on the first LED chip, and configured to emit a light within a visible light band; and a PCB substrate on which the UV LED package is disposed.

### MODE FOR INVENTION

Hereinafter, an embodiment of the present invention will be described in detail with reference to the accompanying drawings so that a person having ordinary skill in the art to which the present invention pertains may easily implement the present invention. However, the present invention may be implemented in various other forms without being limited to embodiments that will be disclosed below. In addition, portions irrelevant to the present invention will be omitted in the drawings in order to clearly disclose the present invention, and the same or similar reference numerals in the drawings will denote the same or similar elements.

The objects and effects of the present invention will be naturally understood or more clearly understood by the following description, and the objects and effects of the present invention are not limited only to the following description.

The objects, features, and advantages of the present invention will become more clearly understood through the following detailed description. In addition, in the following description of the present invention, detailed descriptions of known technologies relevant to the present invention will be omitted when they may make the subject matter of the present invention unnecessarily unclear. Hereinafter, the embodiment of the present invention will be described in detail with reference to the accompanying drawings.

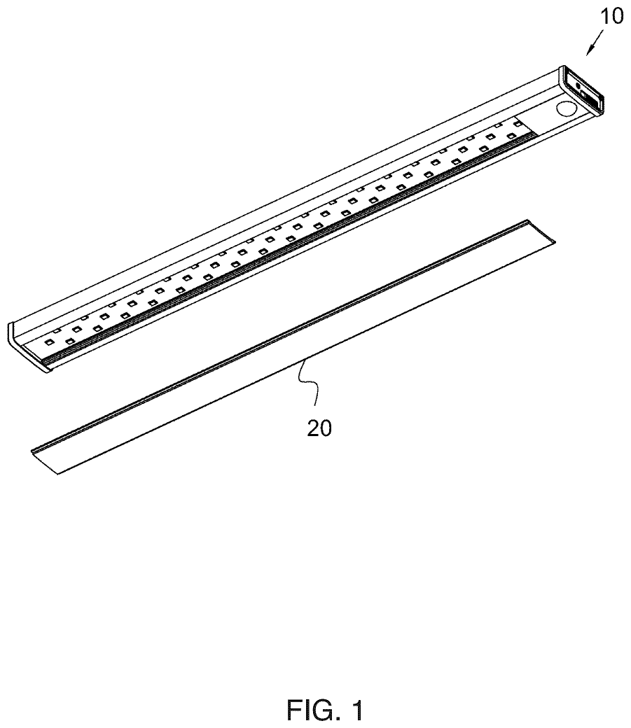

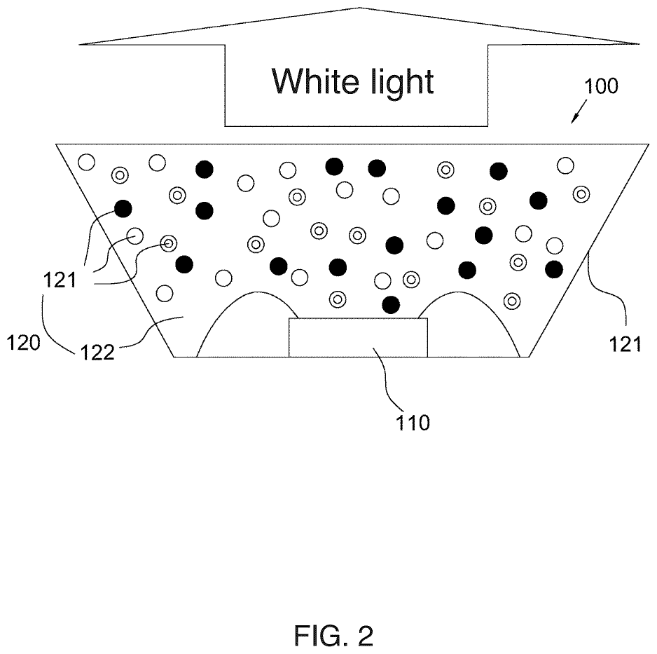

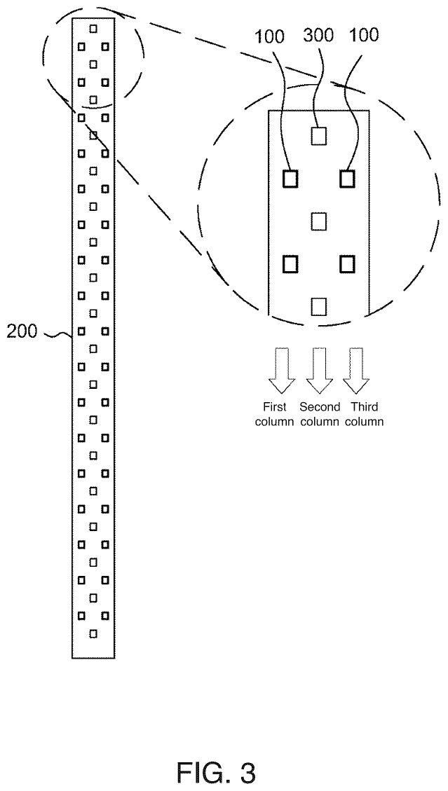

FIG. 1 is a perspective view showing a lighting apparatus 10 according to one embodiment of the present invention, FIG. 2 is a view showing a UV LED package 100 according to one embodiment of the present invention, and FIG. 3 is a view showing the UV LED package 100 and an infrared LED package 300 disposed on a PCB according to one embodiment of the present invention. Referring to FIGS. 1 to 3, a lighting apparatus 10 may include a UV LED package 100, a fluorescent substance 121, an infrared LED package 300, and a PCB substrate 200, the lighting apparatus 10 may further include a cover 20 for covering a surface on which the lighting apparatus 10 emits a light, and the covering of the cover 20 may be performed when not in use for a long time to protect an LED of the lighting apparatus 10 from external foreign substances such as dust. In addition, the covering of the cover 20 may be performed regardless of a use state so that the light may not emitted.

FIG. 2 is a view showing a UV LED package 100 according to one embodiment of the present invention. Referring to FIG. 2, the UV LED package 100 may be configured to emit a light having a center wavelength of 405 nm, and may be provided for a sterilization effect. The UV LED package 100 may include a fluorescent substance layer 120 and a first LED chip 110 to emit the light having the center wavelength of 405 nm, and may be disposed on the PCB substrate 200. In addition, the UV LED package 100 may emit a colored light within a visible light band depending on a configuration of the fluorescent substance layer 120, and may be configured to emit a light having a color such as white, green, orange, or yellow.

The first LED chip 110 may be a diode for emitting a light having a center wavelength of 400 to 410 nm to exhibit a sterilization effect. Since it is most suitable for the sterilization effect when the first LED chip 110 emits the light having the center wavelength of 405 nm, the present invention will be described based on the first LED chip 110 having the center wavelength of 405 nm. The first LED chip 110 may be installed inside the fluorescent substance layer 120 that will be described below to constitute the UV LED package 100 together with the fluorescent substance layer 120.

The fluorescent substance layer 120 may include an epoxy resin 122 and a fluorescent substance 121, and may be configured to allow a light emitted from the first LED chip 110 to be emitted as a light within a visible light band such as a white, green, orange, or yellow light. The fluorescent substance layer 120 may configured to uniformly disperse and apply the fluorescent substance 121. The fluorescent substance layer 120 may be prepared by uniformly dispersing the fluorescent substance 121 in the epoxy resin 122, and may be prepared by applying the fluorescent substance layer 120 onto the first LED chip 110 or placing the fluorescent substance layer 120 on the first LED chip 110 in the form of a thin film, and curing the fluorescent substance layer 120 at 100 to 160° C. for 1 hour. In addition, a paste composition in which the fluorescent substance 121 and a glass frit are mixed with each other may be applied by using a screen printing scheme, so that a small amount of fluorescent substances 121 may be used.

The fluorescent substance 121 may be applied onto the first LED chip 110 so as to be excited by the light emitted from the first LED chip 110 so that the UV LED package 100 may emit various lights within the visible light band including a white light. As described above, the fluorescent substance 121 may be mixed and dispersed with the epoxy resin 122 in the fluorescent substance layer 120 of the UV LED package 100 so as to constitute the UV LED package 100. In this case, the fluorescent substance 121 may include a blue fluorescent substance, a green fluorescent substance, a red fluorescent substance, and a yellow fluorescent substance, and the fluorescent substances 121 for generating colors by using the light emitted from the first LED chip 110 as an excitation source may be combined with each other to produce various lights within the visible light band, such as white, green, orange, and yellow lights.

When the UV LED package 100 is configured as described above to emit various lights within the visible light band including the white light, the UV LED package 100 may emit a colored light exhibiting a sterilization effect, and since the first LED chip 110 having the center wavelength of 405 nm is used in the UV LED package 100, it may be suitable for space sterilization because the wavelength of 405 nm is longer than a wavelength of an ultraviolet light, resulting in a longer sterilization distance and harmlessness to the skin.

Meanwhile, when the UV LED package 100 emits a colored light such as a white light or a green light, the white light or the green light may be emitted by using the fluorescent substances 121 without relatively emitting a blue light as compared with a general case in which sterilization is performed by using a blue LED, so that lighting having a color such as white, green, orange, or yellow, which is not harmful to a human body by minimizing a blue light emitted from lighting, may be used as space lighting.

Therefore, the lighting apparatus 10 according to the present invention may be configured to emit a white light in publicly used facilities requiring a bright white light, the lighting apparatus 10 that emits various color temperatures such as 3000K to 2000K of a white light may be utilized in a place such as a cafe by changing the configuration of the fluorescent substance layer 120, and the lighting apparatus 10 may be utilized as a mood light having a color such as green, orange, or yellow if necessary. When various color temperatures and colors are utilized as described above, the UV LED package 100 capable of performing sterilization may be utilized for the lighting apparatus 10 required in various spaces and places.

The infrared LED package 300 may include a second LED chip for emitting an infrared light having a center wavelength of 845 to 855 nm to assist sterilization, and may exhibit sterilization, drying, and warming effects. Since it is preferable to emit an infrared light having a center wavelength of 850 nm to obtain the above-described effects, one embodiment of the present invention will be described based on a case where the center wavelength is 850 nm.

Since a light emitted from the second LED chip is the infrared light having the center wavelength of 850 nm, the light may not be viewed by a human eye, so that the light may not affect a color of the light emitted from the lighting apparatus 10. Therefore, the infrared LED package 300 may not include the fluorescent substance 121 and the fluorescent substance layer 120, or may include the fluorescent substance 121 and the fluorescent substance layer 120 to emit a colored light. However, manufacture of the infrared LED package 300 including the fluorescent substance layer 120 and the fluorescent substance 121 may be expensive, and the infrared LED package 300 may be disposed together with the UV LED package 100 to utilize a colored light emitted by the UV LED package 100, so that the infrared LED package 300 preferably includes only the second LED chip to emit an infrared light because a cost may be saved.

Meanwhile, the UV LED package 100 may be configured to emit lights having mutually different colors without emitting only a single colored light, and may include a first UV LED package and a second UV LED package, which have mutually different fluorescent substance layers 120 to emit lights having mutually different colors. When the mutually different fluorescent substance layers 120 are provided, a thickness of the fluorescent substance layer 120, a material of the fluorescent substance layer 120, a configuration of the fluorescent substance 121 included in the fluorescent substance layer 120, or the like may be appropriately changed to emit a light within a visible light region, which has a desired color.

When the UV LED package 100 includes the first UV LED package and the second UV LED package, which have the mutually different fluorescent substance layers 120 to emit the lights having the mutually different colors as described above, colored lights emitted from the first UV LED package and the second UV LED package may be combined with each other to emit a light having another color. In addition, although one embodiment of the present invention has been described based on the UV LED package 100 including the first UV LED package and the second UV LED package, which emits lights having two colors, a plurality of UV LED packages 100 for emitting lights having two or more colors may be combined with each other to combine various colored lights so as to emit a specific color, especially a white light that is frequently used in the publicly used facilities.

When the UV LED packages 100 having the mutually different fluorescent substance layers 120 to emit lights having various colors are combined with each other to combine a colored light as described above, more diverse colors and color temperatures may be emitted, so that the present invention may be utilized for various purposes.

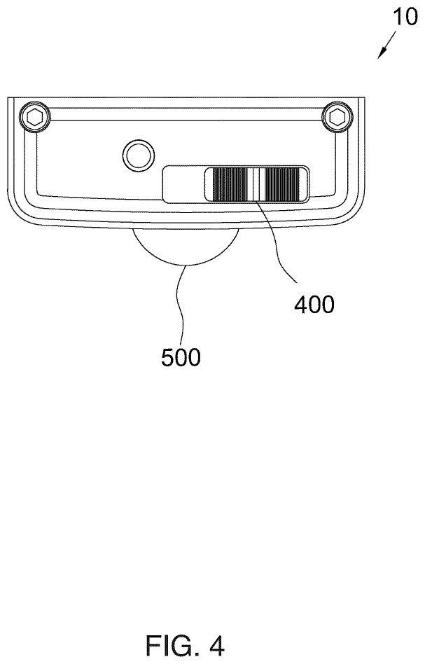

FIG. 4 is a right side view of the lighting apparatus 10 showing a switch 400 according to one embodiment of the present invention. Referring to FIG. 4, the present invention may further include a switch 400, and the switch 400 may serve to cut off or connect a power supplied to the UV LED package 100 and the infrared LED package 300.

The switch 400 may be provided in various types such as button, dip, rocker, rotary, slide, and toggle types. Since the slide type capable of setting various modes is preferable, one embodiment of the present invention will be described based on the slide type. In detail, the switch 400 may serve to turn on all or each of the UV LED package 100 and the infrared LED package 300 or turn off all of the UV LED package 100 and the infrared LED package 300 in the lighting apparatus 10 according to the present invention.

For example, when assuming that there are four positions in which the slide switch 400 may be located, an ON-switch 400 for turning on all lighting, an OFF-switch 400 for turning off all the lighting, a switch 400 for turning on only the UV LED package 100, and a switch 400 for turning on only the infrared LED package 300 may be provided, or an Auto-switch 400 may be further provided to adjust the lighting according to a situation. When the Auto-switch 400 is provided, a motion sensor 500 may be further provided to switch a mode based on a case when a person is present and a case when there is no person.

In this case, in case of using the Auto-switch, lighting of the lighting apparatus 10 may be turned off, the power may be supplied to automatically turn on the lighting when the motion sensor 500 detects a motion, and the power may be automatically cut off to turn off the lighting when the motion sensor 500 does not detect the motion for a predetermined time.

In addition, the UV LED package 100 may be turned off, and turned on when the motion sensor 500 detects the motion, and the infrared LED package 300 may be continuously turned on regardless of whether the motion is detected; or the infrared LED package 300 may be turned on, and turns off when the motion sensor 500 detects the motion, and the UV LED package 100 may be continuously turned on regardless of whether the motion is detected.

In this case, in the case where the UV LED package 100 includes the first UV LED package and the second UV LED package, when the motion sensor 500 detects a motion, one of the first UV LED package and the second UV LED package may be turned off after being turned on, and the remaining one may be turned on after being turned off; and when the motion sensor 500 does not detect the motion for a predetermined time, an operation may be performed opposite to a case when the motion sensor 500 detects the motion so as to return to a state before the motion is detected. When the first UV LED package and the second UV LED package are alternately turned on and off as described above, the lighting may be provided with mutually different colors for a case when a person is present on a periphery and a case when there is no person on the periphery, respectively, so that the present invention may serve as a mood light, a sensor, or the like.

In a case where an operation of turning on or off is performed when the motion sensor 500 detects the motion as described above, when the motion sensor 500 does not detect the motion for the predetermined time, the operation may be performed opposite to the case when the motion sensor 500 detects the motion so as to return to a previous state in which the lighting is turned on or off before the motion is detected. In addition, the predetermined time during which the motion sensor 500 does not detect the motion may be preset or variable if necessary. In this case, although the predetermined time is preferably set to 30 seconds to 1 minute so as to ensure a minimum time for enabling a person to perform a necessary activity in a corresponding space from a time point at which the motion is detected, the predetermined time may be variously set according to a user convenience and purposes.

Meanwhile, the switch 400 is preferably provided on a surface other than the surface on which the lighting apparatus 10 emits the light. In detail, since an opposite surface of the surface on which the lighting apparatus 10 emits the light is generally a surface installed on a wall, a ceiling, or the like, the switch 400 is preferably provided on a side surface of the lighting apparatus 10.

When the switch 400 is provided as described above, the lighting may be adjusted and used in a way desired by a user, so that the lighting may be variously utilized as necessary, and energy may be saved by turning on only the necessary lighting.

FIG. 3 is a view showing the UV LED package 100 and an infrared LED package 300 disposed on a PCB according to one embodiment of the present invention. Referring to FIG. 3, the PCB substrate 200 may be configured such that the UV LED package 100 and the infrared LED package 300 that will be described below are disposed on the PCB substrate 200, and may constitute the lighting apparatus 10.

The UV LED package 100 and the infrared LED package 300 may be installed on the PCB substrate 200, and may be electrically connected to each other through various schemes. For example, the LED chips may be connected in series, in parallel, or in series and parallel, and the PCB substrate 200 may have a circuit pattern for supplying a power to the LED chips. The PCB substrate 200 may be configured as a straight-line, circular, or polygonal plate, and one embodiment of the present invention will be described based on the PCB substrate 200 having a rectangular shape in which one side is long so as to be suitable for a general lighting apparatus 10.

Meanwhile, a wire for supplying the power to the UV LED package 100 and the infrared LED package 300 may be installed on the PCB substrate 200, or the UV LED package 100 and the infrared LED package 300 may be electrically connected to the PCB substrate 200 through soldering so that a separate wire may not be provided. In addition, the UV LED package 100 and the infrared LED package 300 may be electrically connected to the PCB substrate 200 by a surface-mount technology (SMT).

Meanwhile, the UV LED package 100 and the infrared LED package 300 may be disposed on the PCB substrate 200. Since the UV LED package 100 serves to emit the light within the visible light band including the white light as described above, it is preferable to arrange more UV LED packages 100 than infrared LED packages 300. FIG. 3 is a view showing the UV LED package 100 and an infrared LED package 300 disposed on a PCB according to one embodiment of the present invention. Referring to FIG. 3, according to one embodiment of the present invention, three columns may be configured based on a long side of the PCB board 200, in which the UV LED package 100 is arranged in a first column and a third column, which are both side columns, and the infrared LED package 300 is arranged in a second column, which is a middle column; the UV LED packages 100 of the both side columns may be located at regular intervals in the same row in each of the columns of the UV LED package 100; and the infrared LED package 300 of the second column, which is the middle column, may be arranged between rows of the UV LED packages 100 arranged in the first column or the second column, so that the UV LED package 100 and the infrared LED package 300 may be alternately arranged with each other, thereby reducing an interference and ensuring a balanced arrangement. Meanwhile, various other arrangements that may serve as lighting are also possible, and efficiency or a role of the lighting may vary depending on the arrangements.

In addition, when the UV LED package 100 includes the first UV LED package and the second UV LED package as described above, the first UV LED package may be arranged in the first column, and the second UV LED package may be arranged in the third column; the above arrangement may be performed by conversely changing the columns; and the first UV LED package and the second UV LED package may be mixedly arranged in each of the columns, in which it is preferable to alternately arrange the first UV LED package and the second UV LED package once in each of the columns so that two colors may be properly mixed. When the UV LED package 100 includes the first UV LED package and the second UV LED package that emit mutually different colored lights as described above, the first UV LED package and the second UV LED package may be disposed together on the PCB substrate 200 to emit a light having a mixed color. Next, a sterilization effect of the PCB substrate 200 into which the UV LED package 100 and the infrared LED package 300 are inserted will be observed through a first antibacterial test and a second antibacterial test.

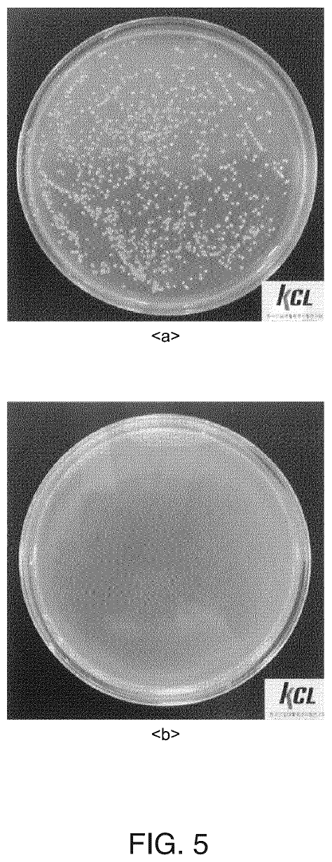

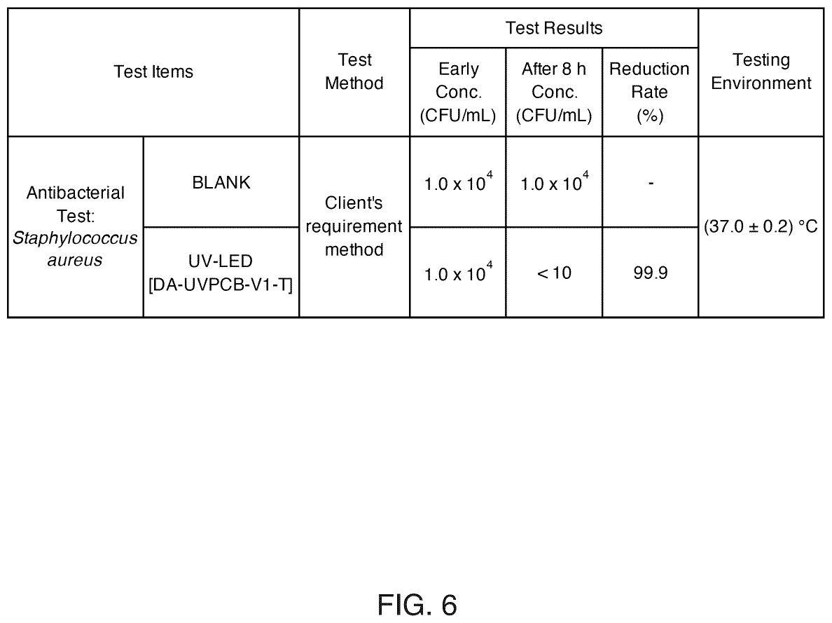

FIG. 5 is a photograph showing results of a first antibacterial test of a PCB substrate 200 into which the UV LED package 100 and the infrared LED package 300 are inserted according to the present invention, and FIG. 6 is a table showing results of the first antibacterial test of the PCB substrate 200 into which the UV LED package 100 and the infrared LED package 300 are inserted according to the present invention. Referring to FIGS. 5 and 6, a test strain of the first antibacterial test was Staphylococcus aureus ATCC 6538, and a reduction rate was measured by exposing a medium inoculated with the test strain at a distance of 80 mm from the PCB substrate 200 into which the UV LED package 100 and the infrared LED package 300 are inserted. FIG. 5<a> is a photograph after 0 hour, and FIG. 5<b> is a photograph after exposure of 8 hours, in which it may be found with a naked eye that the test strain is sterilized and disappeared. Referring to FIG. 6, it may be found that a sterilization effect of 99.9% is obtained after 8 hours.

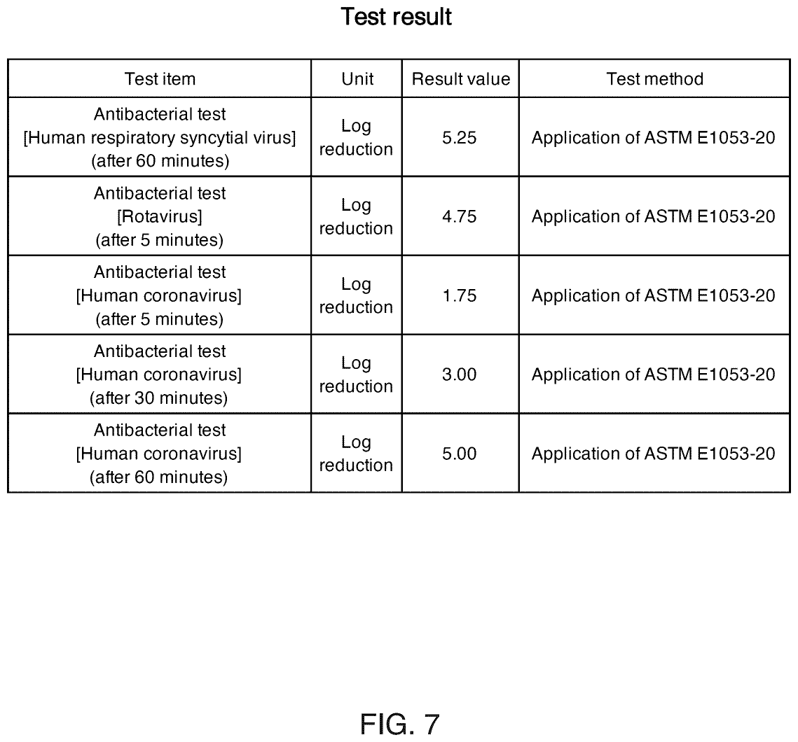

FIG. 7 is a table showing results of a second antibacterial test of the PCB substrate 200 into which the UV LED package 100 and the infrared LED package 300 are inserted according to the present invention. Referring to FIG. 7, the second antibacterial test is an antibacterial test of the PCB substrate 200 into which the UV LED package 100 and the infrared LED package 300 are inserted for a human respiratory syncytial virus, Rotavirus A, and a human coronavirus. Data was measured over time by irradiating the virus at a distance of 15 mm from the PCB substrate 200 into which the UV LED package 100 and the infrared LED package 300 are inserted, in which a sterilization effect of 99.99% or more is obtained when a log reduction value is 4 or more, and a sterilization effect of 99.999% or more is obtained when a log reduction value is 5 or more. When the irradiation is performed with the PCB substrate 200 into which the UV LED package 100 and the infrared LED package 300 are inserted for 60 minutes, it may be found that a sterilization effect of 99.999% or more is obtained because a log reduction value of each of the respiratory syncytial virus and the human coronavirus is 5 or more, and it may be found that a sterilization effect of 99.99% or more is obtained because a log reduction value acquired as a result value of the rotavirus for 60 minutes is 4 or more. When the PCB substrate 200 into which the UV LED package 100 and the infrared LED package 300 are inserted, which is verified to have the sterilization effect, is used as the lighting as described above, the sterilization effect may be continuously exhibited.

The preferred embodiment of the present invention described above is disclosed for illustrative e purposes, various modifications, changes, and additions can be made by those of ordinary skill in the art without departing from the idea and scope of the present invention, and such modifications, changes, and additions are to be considered as falling within the scope of the appended claims.

Since various substitutions, modifications, and changes can be made by a person having ordinary skill in the art to which the present invention pertains without departing from the technical idea of the present invention, the present invention is not limited by the above-described embodiments and the accompanying drawings.

Although methods have been described based on a flowchart as a series of steps or blocks in an above-described exemplary system, the present invention is not limited to an order of the steps, and some steps may occur in a different order or simultaneously with a step other than the steps described above. In addition, it is to be understood by those skilled in the art that the steps shown in the flowchart are not exclusive, in which other steps may be included, or one or more steps in the flowchart may be deleted without affecting the scope of the present invention.

### INDUSTRIAL APPLICABILITY

When the lighting apparatus according to the present invention is used, while lighting having various colors within the visible light band, such as white, green, orange, and yellow, and color temperatures illuminates an inside of a room, a damage caused by a blue light can be reduced, a sterilization effect can be obtained, and a COVID-19 virus can be effectively killed. Since there is always lighting in public transportation and publicly used facilities, which are usually crowded by the public, when the lighting installed in the public transportation and the publicly used facilities is replaced with the lighting apparatus according to the present invention, the sterilization effect may be naturally achieved while the lighting is provided, so that the spread of various pathogens and the COVID-19 virus can be reduced.

1. A lighting apparatus (10) comprising:
at least one UV LED package (100) including a first LED chip (110) for emitting a light having a center wavelength of 400 to 410 nm and a fluorescent substance layer (120) formed on the first LED chip (110), and configured to emit a light within a visible light band; and
a PCB substrate (200) on which the UV LED package (100) is disposed.

2. The lighting apparatus (10) of claim 1, further comprising at least one infrared LED package (300) including a second LED chip for emitting a light having a center wavelength of 845 to 855 nm.

3. The lighting apparatus (10) of claim 1, wherein the light within the visible light band includes a light having one of a white color, a green color, an orange color, and a yellow color.

4. The lighting apparatus (10) of claim 2, wherein the UV LED package (100) includes a first UV LED package and a second UV LED package, which have mutually different fluorescent substance layers (120) to emit lights having mutually different colors.

5. The lighting apparatus (10) of claim 4, further comprising a switch (400) serving to cut off or connect a power supplied to the UV LED package (100) and the infrared LED package (300).

6. The lighting apparatus (10) of claim 5, further comprising a motion sensor (500),
wherein the switch (400) further includes an Auto-switch for operating the motion sensor (500) to perform an operation according to motion detection of the motion sensor (500).

7. The lighting apparatus (10) of claim 6, wherein, in a case of using the Auto-switch,
the power is supplied to automatically turn on lighting when the motion sensor (500) detects a motion, and
the power is automatically cut off to automatically turn off the lighting when the motion sensor (500) does not detect the motion for a predetermined time.

8. The lighting apparatus (10) of claim 6, wherein, in a case of using the Auto-switch,
when the motion sensor (500) detects a motion, the first UV LED package is turned off after being turned on, and the second UV LED package is turned on after being turned off; and
when the motion sensor (500) does not detect the motion for a predetermined time, the first UV LED package is turned on again, the second UV LED package is turned off again, and the infrared LED package (300) is continuously turned on regardless of whether the motion is detected.

9. The lighting apparatus (10) of claim 6, wherein, in a case of using the Auto-switch,
the infrared LED package (300) is turned on, and turned off when the motion sensor (500) detects a motion,
the infrared LED package (300) is turned on again when the motion sensor (500) does not detect the motion for a predetermined time, and
the UV LED package (100) is continuously turned on regardless of whether the motion is detected.

10. The lighting apparatus (10) of claim 4, wherein the PCB substrate (200) is configured such that the UV LED package (100) and the infrared LED package (300) are arranged in three columns;
the UV LED package (100) is arranged in a first column and a third column, and the infrared LED package (300) is arranged in a second column; and
the UV LED packages (100) arranged in the first and third columns are arranged in a same row, and the infrared LED package (300) of the second column is arranged between rows of positions where the UV LED packages (100) of the first and third columns are arranged so as to be alternately arranged with the UV LED packages (100) of the first and third columns.
